(19) United States
(12) Patent Application Publication
(10) Pub. No.: US 2003/0113723 A1
(43) Pub. Date: Jun. 19, 2003

(54) Method for evaluating microsatellite instability in a tumor sample

(76) Inventors: Bharati Bapat, Toronto (CA); Melanie Anne Rose, Georgetown (CA)

Correspondence Address: MERCHANT & GOULD PC, P.O. BOX 2903, MINNEAPOLIS, MN, 55402-0903, US

(21) Appl. No.: 09/971,353

(22) Filed: Oct. 4, 2001

Related U.S. Application Data
(60) Provisional application No. 60/237,884, filed on Oct. 4, 2000.

(51) Int. Cl. C12Q 1/68; C12P 19/34

(52) U.S. Cl. 435/6; 435/91.2

(57) ABSTRACT

A detection method for high throughput screening for tumor microsatellite instability. The method employs a panel of microsatellite loci and it is based on a fluorescent multiplex PCR system. The method provides a fast, sensitive, and cost-effective high throughput screening method of MSI detection. The method allows many samples to be processed in one day on a single polyacrylamide gel, and it utilizes much less nucleic acid sample than conventional methods.

### FIELD OF THE INVENTION

The invention relates to the evaluation of microsatellite instability in a tumor sample by detecting microsatellite loci in the sample.

### BACKGROUND OF THE INVENTION

Microsatellite instability (MSI) is defined as the occurrence of novel alleles in tumor DNA with a frequency of at least 40% among microsatellite loci examined. Defects in the mismatch repair system causes MSI which plays an important role in the development of tumors. MSI was first reported in colorectal tumors (Peinado, M. A. et al, 1992 Proc. Natl. Acad, Sci. USA 89: 10065-69; Ionov, Y. Nature (London) 363:558-61; Thibodeau, S N. Et al Science 260, 1993 260: 816-819), and later in several other tumor types (Risinger, J I Cancer Res, 1993,53:5100-03; Han, H J et al Cancer Res 1993 Cancer Res 1993, 53:5087-89; Peltomaki, P, 1993, 53:5853-55; Gonzalez-Zulueta, M et al Cancer Res 1993, 5620-23; Merlo, A et al Cancer Res 1994, 54:2098-2101). MSI in inherited nonpolyposis colorectal carcinoma in patients are apparently due to inherited and somatic mutations in mismatch repair genes (Leach F et al, 1993, 75: 1215-1225; Fishel R et al, 1993, Cell 75: 1027-38; Papadopoulos, N, et al 1994, Science 263: 1625-29, 1994; Bronner, C. E. et al, 1994, Nature (London) 368:258-61 (1994).

Detection of tumors with MSI has important prognostic and treatment implications for patients. For example, microsatellite markers have been used for colon cancer detection (Cawkwell et al, 1994, Br. J. Cancer 70:813-18). PCR is used for identifying both the appearance of new polymorphisms and the loss of heterozygosity in cancer detection (Mao, L. et al Proc. Natl. Acad. Sci. 1994, 91: 9871-75; Mao, L et al, 1996 Science, 271:659-62; Radford, et al Cancer Res., 1995, 55:3399-05). However, PCR has limitations in that each PCR reaction is run individually and separated on a sequencing gel.

There is a need for large-scale multiplex methods for detecting large numbers of microsatellite loci for practical identification of individuals for genetic cancer diagnosis and prognosis.

### SUMMARY OF THE INVENTION

The present inventors have developed a MSI detection method for high throughput screening for tumor microsatellite instability. The method employs a panel of microsatellite loci and it is based on a fluorescent multiplex PCR system and automated fragment analysis. The method provides a fast, sensitive, and cost-effective high throughput screening method of MSI detection. The protocols described herein are simple enough to be performed in a routine clinical laboratory. The method also allows many samples to be processed in one day on a single polyacrylamide gel, and it utilizes much less nucleic acid sample (about 25ng) than conventional methods.

Broadly stated the present invention relates to a method for evaluating microsatellite instability in a tumor sample by detecting microsatellite loci in the sample comprising:

(a) amplifying in the sample at least two selected microsatellite loci associated with cancer to provide labeled amplified products or amplicons that are complementary to microsatellite loci sequences in the tumor sample;

(b) detecting the labeled amplified products or amplicons and distinguishing the amplified products to indicate the presence of one or more of the microsatellite loci in the sample; and

(c) repeating steps (a) and (b) for at least two different selected microsatellite loci.

In an embodiment of the invention the microsatellite loci are amplified using a multiplex polymerase chain reaction.

In an embodiment, a method is provided for evaluating microsatellite instability in a tumor sample by detecting microsatellite loci in the sample comprising:

(a) forming a polymerase chain reaction mixture comprising the tumor sample, a polymerase, and primer sets for at least two selected microsatellite loci associated with cancer, each primer set characterized by (a) a forward primer containing a sequence complimentary to a 5′ upstream primer-specific portion of a selected microsatellite loci; and (b) a reverse primer complementary to a 3′ downstream primer-specific portion of the same microsatelite loci, wherein one of the primers has a detectable reporter label;

(b) subjecting the polymerase chain reaction mixture to polymerase chain reaction cycles to form amplified products complementary to microsatellite loci in the tumor sample;

(c) detecting the reporter labels and distinguishing the amplified products to indicate the presence of one or more of the microsatellite loci in the sample; and

(d) repeating steps (a) to (c) with primer sets for at least two different selected microsatellite loci.

The invention also contemplates kits comprising compositions selected from the group consisting of primers and ancillary reagents used in an amplification reaction (preferably PCR) in a method for evaluating microsatellite instability in a tumor sample.

The methods ofthe invention may be used to determine a genomic instability index. The index may be calculated as follows:

(# alterations in the banding pattern from the amplified tumor cell DNA/total number of bands in the pattern from the amplified normal cell DNA)×100

The methods of the present invention may be used to detect cancer, particularly cancers involving defects in mismatch repair. Various aspects of the invention may be used to identify defects in mismatch repair of genes in the following human cancers: leukemia, colorectal cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors and cervical cancer.

The methods of the present invention have particular application in the diagnosis and monitoring of colorectal cancer. MSI is observed in approximately 15-25% of sporadic colorectal cancers and more than 85% of colorectal cancers arising in patients with hereditary non-polyposis colorectal cancer syndrome.

Therefore, the present invention provides a method for diagnosing colorectal cancer or hereditary non-polyposis colorectal cancer syndrome in a human individual comprising the steps of (a) isolating DNA from the human individual; (b) assaying the DNA using multiplex PCR for microsatellite loci associated with colorectal cancer or hereditary non-polyposis colorectal cancer syndrome relative to a normal human individual (c) diagnosing colorectal cancer or hereditary non-polyposis colorectal cancer syndrome in the human individual based on the frequency of microsatellite loci. In an aspect of the invention the microsatellite loci that are assayed include BAT26, D17S250, MYC-L, one or both of BAT40, and optionally one or more of BAT25, D5S346, D2S123, ACTC, D10S197 and D18S55. In aparticular embodiment ofthe invention two PCR reactions are used to assay the microsatellite loci, and the amplicons or extension products are analyzed using automated fragment analysis. The diagnostic method facilitates a determination of the optimum treatment regimen for the individual.

In an embodiment of a method of the invention at least 2, preferably at least 4, 6, 8, or 10 microsatellite loci are detected. In a particular embodiment 11 microsatellite loci are detected.

The method of the invention enables the identification of different types of tumors (e.g. colorectal tumor and other tumors) including MSI-H tumors (high or >40% frequency of MSI among the panel of microsatellite loci, i.e. MSH+), MSI-L tumors (low or <40% of MSI), and MSS (microsatellite stable) tumors (Boland, R et al, Cancer Res. 58:5248-570). LOH tumors may also be identified using a method of the invention. LOH or “Loss of Heterozygosity” refers to an allelic imbalance where an allele is lost/reduced in the tumor when compared with its expression in matched normal cells.

In colorectal cancer, individuals with MSI-H tumors have a better outcome than those with MSI-L or MSS tumors. MSI-High indicates a change in the mismatch repair pathway with probable inactivation of the mismatch repair genes, hMSH2 or hMLH1. Further screening of hMSH2 or hMSLH1 may be carried out, and where there is a strong family history germline mutation screening of mismatch repair genes may be undertaken. In individuals where there is no family history, hypermethylation of the hMLH1 promoter region may be analyzed.

These and other aspects, features, and advantages of the present invention should be apparent to those skilled in the art from the following drawings and detailed description.

### BRIEF DESCRIPTION OF THE DRAWINGS

The invention will now be described in relation to the drawings in which:

FIG. 1 is a schematic diagram of a method of the invention.

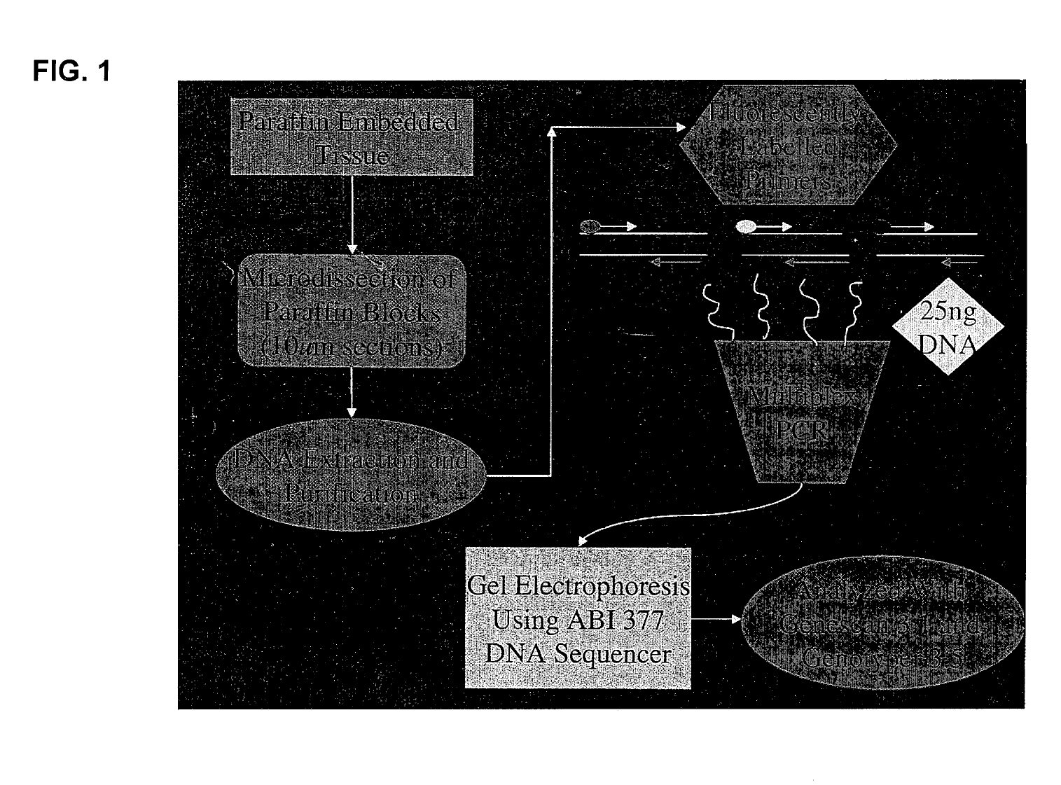

FIG. 2 shows sensitivity detection of MSI at mononucleotide repeats and MSI at dinucleotide repeats.

FIG. 3 is a comparison of a MSI analysis of mononucleotide repeats of a colorectal tumor and a normal subject using an automated multiplex method of the invention and a manual radioactive method.

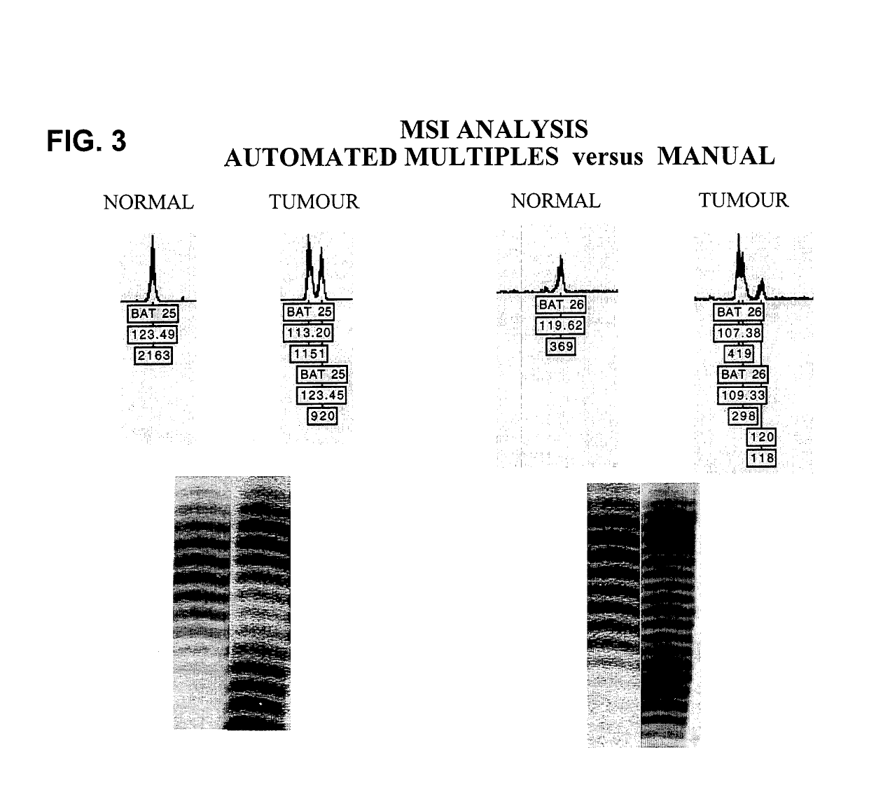

FIG. 4 is a comparison of a MSI analysis of dinucleotide repeats of a colorectal tumor and a normal subject using an automated multiplex method of the invention and a manual radioactive method.

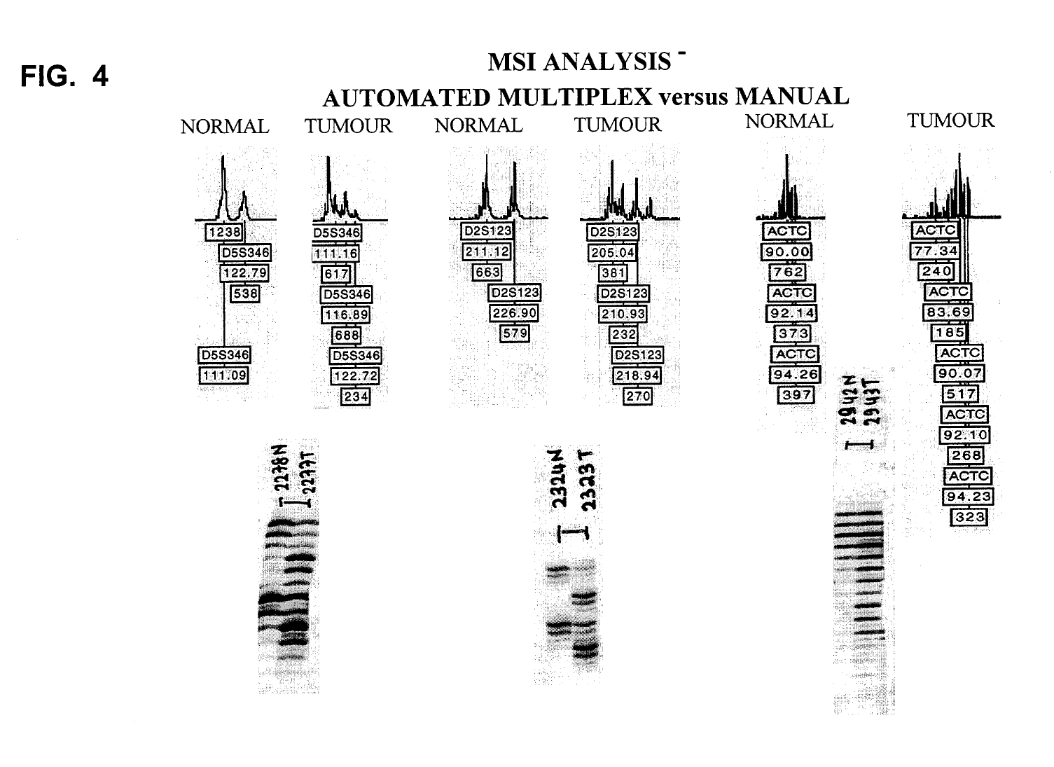

FIG. 5 shows the results of an analysis of MSI-High tumor with the panel 1 loci (Table 1).

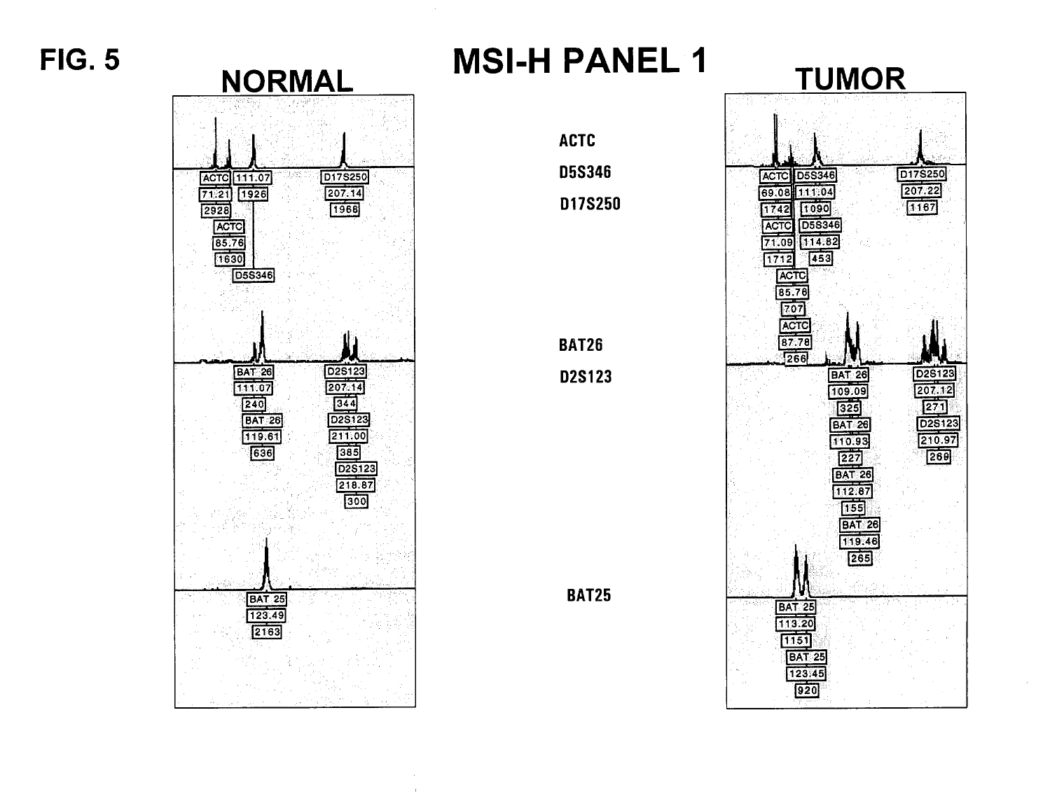

FIG. 6 shows the results of an analysis of MSI-High tumor with the panel 2 loci (Table 2).

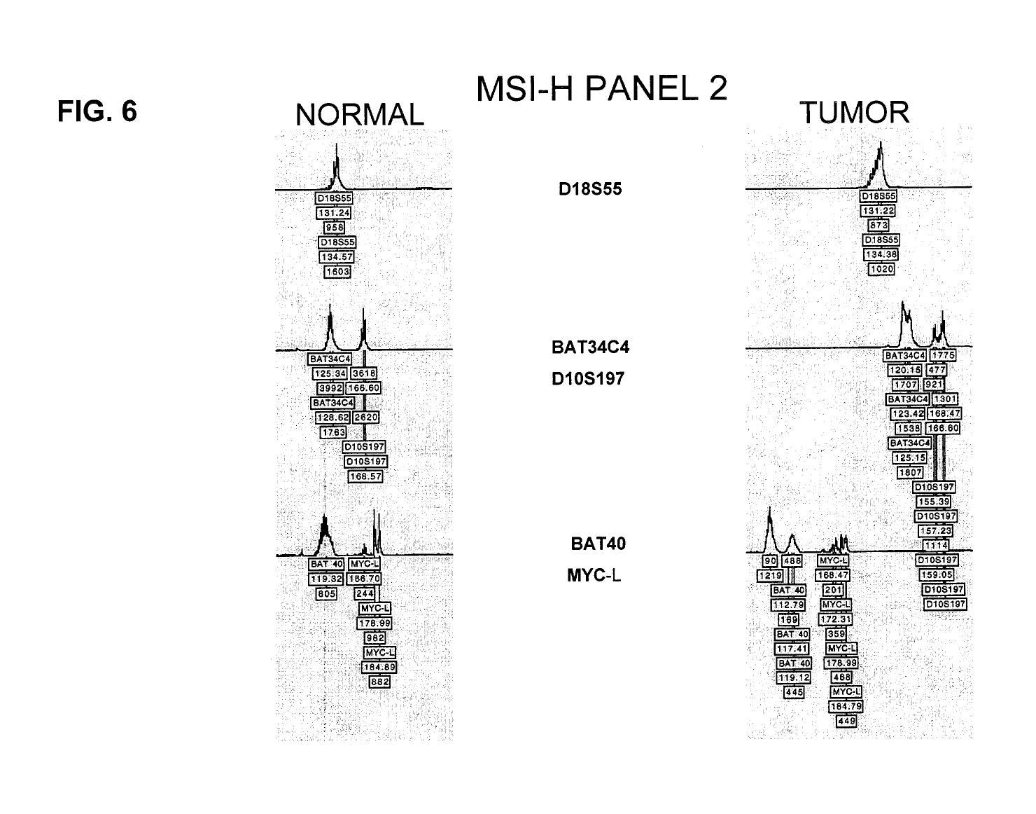

FIG. 7 shows the results of an analysis of a LOH tumor with panel 1 loci (Table 2).

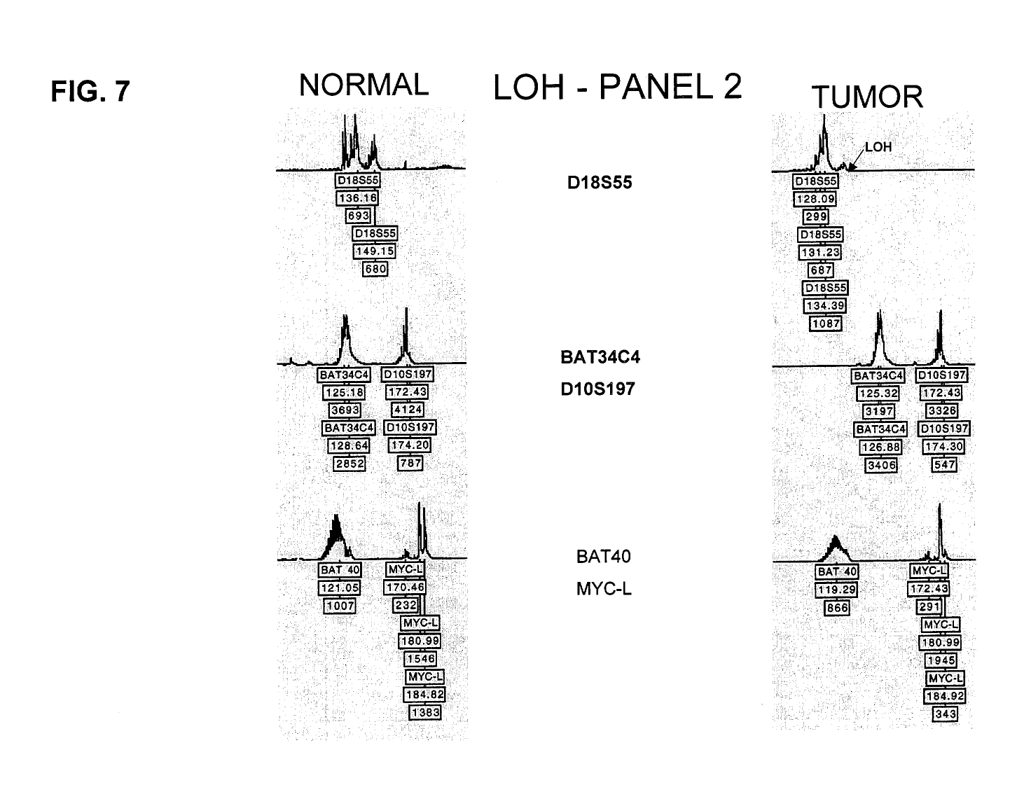

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Glossary

The term “individual” refers to any mammal, particularly humans.

The term “amplicon” or “amplified product” refers to a discreet amplification product synthesized in an amplification reaction (e.g. PCR reaction) and corresponding regions that is intended to be amplified in a method of the invention.

The term “microsatellite instability” or “microsatellite analysis” refers to the measurement or detection of alterations in microsatellite sequences which are known to those skilled in the art to represent a specific pattern of genomic instability caused by DNA mismatch repair defects. Alterations in microsatellite sequences are clinically useful in the diagnosis and monitoring of various types of cancer. Microsatellites are short tandem repeat sequences that are broadly distributed in a genome.

The term “amplify” or “amplification” refers to a process by which nucleotide sequences are amplified in number. There are several well known means for enzymatically amplifying nucleotide sequences (see review in BioTechnology 8:290-3, 1990). The most commonly used method is the Polymerase Chain Reaction (PCR). PCR employs a thermostable DNA polymerase, known primer sequences, and heating cycles that separate the replicating DNA strands and exponentially amplify a nucleotide sequence of interest. The PCR process is fully described in Erlich et al, Science 1991 252:1643-50, M. Innis, et al Science 1988, Science 239:

487-91). Other amplification systems include ligase chain reaction (LCR), and nucleic acid sequence-based amplification (NASBA). The invention is not limited to any particular amplification system, since other systems may be developed which would benefit by the practice of the invention.

A “mulitplex polymerase chain reaction” is a polymerase chain reaction wherein more than one region of target DNA is amplified simultaneously in a single reaction vessel.

The term “detectable reporter label” or “label” refers to a molecule that is incorporated indirectly or directly into an oligonucleotide primer of the amplified product. A label molecule facilitates the detection of an oligonucleotide which becomes part of an amplified DNA sequence. Suitable labels include chromophores, fluorescent molecules, enzymes, antigens, heavy metals, magnetic probes, dyes, radioactive materials, phosphorescent groups, chemiluminescent moieties, and electrochemical detecting moieties.

A label may be incorporated directly into an oligonucleotide by using a deoxynucleoside triphosphate (dNTP) containing a label in the process of synthesizing the oligonucleotide. Alternatively, the label may be incorporated indirectly into an oligonucleotide by coupling a primer at the 5′ end with a linker (e.g. aminohexyl linker) using a standard DNA synthesis cycle and coupling a label such as a fluorescent dye-NHS ester via the linker. Fluorescent molecules that are useful for labeling an oligonucleotide are known to those skilled in the art. Such molecules may include amine-reactive groups that are reactive to end terminal amines of an oligionucleotide, sulfonyl chlorides that are conjugated to an oligonucleotide through amine residues, and like molecules. A fluorescent molecule may be attached by covalent or non-covalent means. Protocols for incorporating fluorescent molecules are described for example in Kamik, et al, 1995 Hum. Mol. Genet. 4:1889-1894.

The term “primer” refers to an oligonucleotide capable of acting as a point of initiation for DNA synthesis when annealed to a complimentary sequence under suitable conditions, and in the presence of nucleotide triphosphosphates. The primers can be in the form of ribonucleotides, deoxyribonucleotides, modified ribonucleotides, modified deoxyribonucleotides, modified phosphate-sugar backbone oligonucleotides, nucleotide analogs, and mixtures thereof.

The term “sample” refers to any body tissue or fluid suitable for detecting tumor cells, including biopsies, bone marrow aspirates, lymph node aspirates, effusions, ascites, cerebrospinal fluid, and peripheral blood. The sample is preferably a nucleic acid sample from the above tissues or fluids suitable for detecting tumor cells. Methods for preparing nucleic acid samples are well known to those skilled in the art. The concentration of nucleic acids in the samples to be used in the present invention may be about 20-75 ng, more preferably 25 to 50 ng, most preferably 20-25 ng.

In the amplification steps of the present invention a PCR reaction may be employed which utilizes primer sets for at least four (preferably at least 6, 8, or 10) selected microsatellite loci associated with cancer, particularly mismatch-repair deficient tumors, preferably sporadic colorectal and hereditary non-polyposis colorectal cancer syndrome. The primers are selected so that they are suitable for hydridization on complementary strands of a corresponding target microsatellite loci to permit formation of a polymerase chain reaction extension products. There is a mismatch which interferes with formation of such an extension product when the primers hybridize to any other nucleotide sequence present in the sample. The concentration of the forward primers in the PCR reaction mixture may be about 25 to 65 ng, and the concentration of the reverse primers in the PCR reaction mixture may be about 30 to 840 ng. The PCR extension products in a particular set may be distinguished from other PCR extension products in different sets. The primers are blended with the sample to form a polymerase chain reaction mixture. The mixtures are subjected to one or more polymerase chain reaction cycles involving a denaturation treatment, a hydridization treatment, and an extension treatment. In the hybridization treatment the target specific portion of a primer is hybridized to the target microsatellite loci. In the extension treatment, the hybridized primers are extended to form extension products complementary to the target nucleotide sequence to which the primer is hybridized. Detailed process conditions for carrying out the amplification steps are set out in the protocols in the Example.

After the reaction mixture is subjected to the PCR cycles, the labeled extension products are detected. This indicates that presence of one or more target microsatellite sequences in the sample. Automated fragment analysis may be used to detect the labeled fragments.

In accordance with an aspect of the invention, the first amplification (e.g. PCR) step employs primers for the BAT26 and D17S250 loci, and optionally one or more of BAT25, D5S346, D2S123, and ACTC. In accordance with another aspect of the invention, the second amplification step employs primers for MYC-L, one or both of BAT40 and BAT34C4, and optionally one or both of D10S197 and D18S55. Specific microsatellite primer pairs used to amplify and detect microsatellite instability in accordance with the invention are disclosed in Tables 1 and 2 attached hereto. General Information on the loci is set out in Table 5.

The following non-limiting examples are illustrative of the present invention:

### EXAMPLE 1

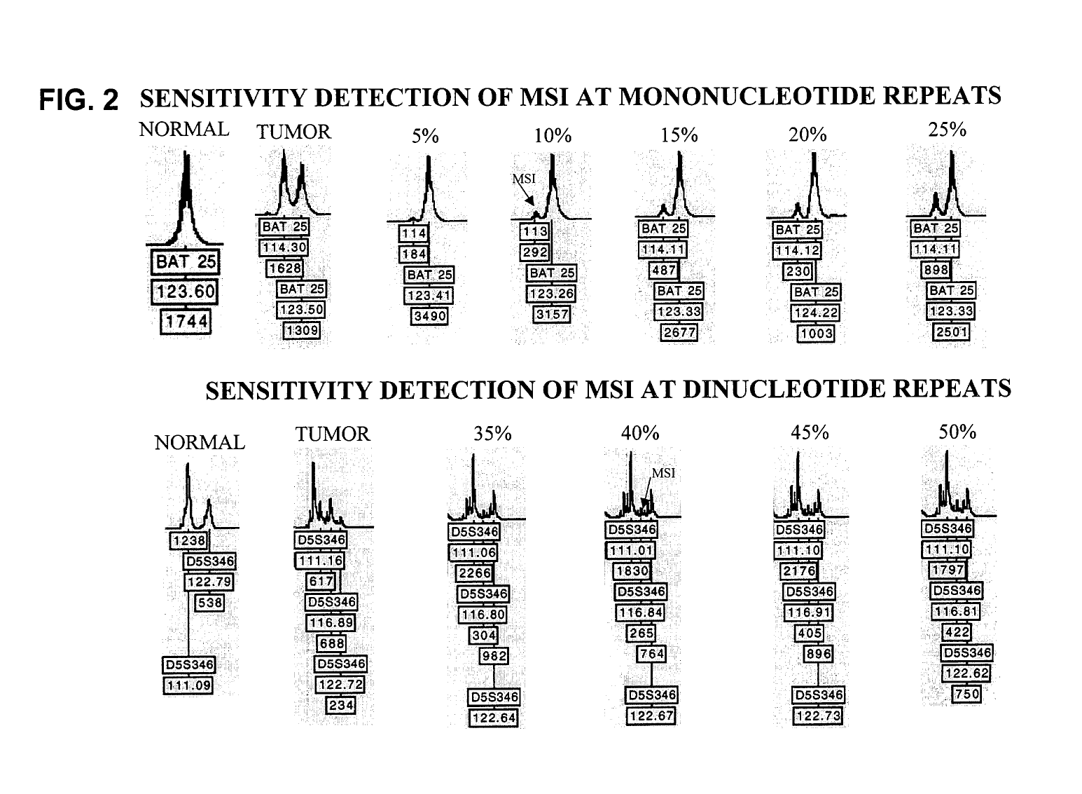

An efficient diagnostic test was developed for screening of tumor MSI based on a fluorescent multiplex PCR system and automated fragment analysis. The assay consists of a panel of 11 microsatellite loci including those loci (BAT25, BAR26, D2S123, D5S346 and D17S250) recommended by the National Cancer Institute (Boland R et al, 1997 Cancer Res 58:5248-57). The microsatellite panel includes one tetranucleotide, six dinucleotide, and four mononucleotide loci. DNA extracted from paraffin embedded tissue (25ng) is amplified in two multiplex PCR reactions. (See detailed protocols set out below and Tables 3 (1^{st} amplification step) and Table 4 (2^{nd} amplification step) setting out various PCR reactions that were carried out to determine the optimal conditions for a method of the invention.) The fluorescent labelled PCR amplicons (size range 65-230 bp) are analyzed using AB1377 GeneScan and Genotyper software. This diagnostic assay was validated by analyzing ten colorectal cancer cases by both fluorescent multiplex PCR and conventional radioactive labelled PCR and gel electrophoresis. The assay sensitivity was determined by MSI analysis of tumor DNA serially diluted with matched normal DNA, and was found to range from 10% for mononucleotide loci to 40% for dinucleotide loci (FIG. 2). Overall, this diagnostic assay offers a fast, sensitive, and cost-effective method of MSI detection and is most suitable for high throughput screening for mismatch-repair deficient tumors.

Microsatellite Analysis Protocols

1^{ST} Amplification Step

Normal and Tumor DNA samples arrive from the Biospecimen repository already purified using the Qiagen Tissue Kit.

1.) Dilute DNA to 25 ng from the original concentration.

2.) STEP 1 PCR: Set up master mix cocktail with the following conditions:

3.0 ul 10X PCR Buffer

0.9 ul 50X MgCl (1.5 mM final)

0.6 ul Forward Primer D17S250

0.6 ul Reverse Primer D17S250

1.2 ul dNTP's (0.4 mM final)

0 ul H₂O

0.6 ul Taq (3 units final)

2.) To each labelled tube add the appropriate DNA (normal or tumor) in the following format:

1.0 ul DNA (25 ng final)

2.0 ul H₂O

3.) With tubes on ice, add 6.9 ul of master mix cocktail to each tube and pipette up and down a few times to mix.

4.) PCR tubes at the following conditions:

| | |
|---|---|
| 94° C. | 5 min |
| 94° C. | 30 sec - - - 40 cycles |
| 55° C. | 30 sec - - - 40 cycles |
| 72° C. | 30 sec - - - 40 cycles |
| 72° C. | 10 min |
| 4° C. | forever |

5.) STEP 2 PCR: While the PCR reaction is starting mix the remaining primers in a tube in the following amounts:

0.3 ul Forward Primers of ACTC, D5S346 (33 ng each primer)

0.6 ul Forward Primers of D2S123, BAT 26, BAT 25 (66 ng each primer)

0.3 ul Reverse Primer of ACTC (33 ng each primer)

0.6 ul Reverse Primer of BAT 26 (66 ng each primer)

4.2 ul Reverse Primers of D2S123 and D5S346 (420 ng each primer)

8.4 ul Reverse Primer BAT 25 (840 ng each primer)

6.) Add 20.1 ul of above mixture to each of the tubes when the reaction has gone through 5 cycles. Continue with PCR program.

7.) Dilute PCR product using 7 ul PCR product mixed with 16 ul H₂O

8.) Make a master mix of loading dye, formamide and TAMRA in the following amounts:

1X

4.0 ul formamide

1.0 ul Genescan TAMRA

0.5 ul loading dye

9.) Mix 4.0 ul of diluted PCR product with 5.5 ul of loading dye mix.

10.) Load 2.0 ul of above unto a 5% polyacrylamide gel and run for 2 hours on the ABI 377 Sequencer (3000 volts, 60 milliAmps and 200 Watts).

11.) Analyze data using GeneScan and Genotyper.

2^{ND} Amplification Step

Normal and Tumor DNA samples arrive from the Biospecimen repository already purified using the Qiagen Tissue Kit.

1.) Dilute DNA to 25 ng from the original concentration.

2.) Set up master mix cocktail with the following conditions:

2.0 ul 10X PCR Buffer

0.6 ul 50X MgCl (1.5 mM final)

1.5 ul Forward Primer (0.3 μl each of BAT 40, MYC-L, BAT 34C4, D10S197, D18S55) (33 ng each primer)

1.5 ul Reverse Primer (0.3 ul each of BAT 40, MYC-L, BAT34C4, D10S197, D18S55) (33 ng each primer)

0.8 ul dNTP's (0.4 mM final)

8.2 ul H₂O

0.4 ul Taq (2 units final)

2.) To each labelled tube add the appropriate DNA (normal or tumor) in the following format:

1.0 ul DNA (25 ng final)

4.0 ul H₂O

3.) With tubes on ice, add 25 ul of master mix cocktail to each tube and pipette up and down a few times to mix.

4.) PCR tubes at the following conditions:

| | |
|---|---|
| 94° C. | 5 min |
| 94° C. | 30 sec - - - 40 cycles |
| 57° C. | 30 sec - - - 40 cycles |
| 72° C. | 30 sec - - - 40 cycles |
| 72° C. | 10 min |
| 4° C. | forever |

5.) Dilute PCR product using 7 ul PCR product mixed with 16 μl H₂O

6.) Make a master mix of loading dye, formamide and TAMRA in the following amounts:

1X

4.0 ul formamide

1.0 ul Genescan TAMRA

0.5 ul loading dye

7.) Mix 4.0 ul of diluted PCR product with 5.5 ul of loading dye mix.

8.) Load 2.0 ul of above unto a 5% polyacrylamide gel and run for 2 hours on the ABI 377 Sequencer (3000 volts, 60 milliAmps and 200 Watts).

9.) Analyze data using GeneScan and Genotyper

### Example 2

Below is a summary of colorectal cancer cases analyzed using a multiplex MSI panel and their MSI status.

| PANEL | MSI-H | MSI-L | MSS | TOTAL |
|---|---|---|---|---|
| I (6 loci) | 76 | 2 | 170 | 248 |
| II (11 loci) | 2 | 12 | 0 | 14 |

TABLE 1
First Panel of Markers for Evaluation of MSI in Colorectal Cancer

| | | PRODUCT | MONO OR | 5′ FLUORESCENT |
|---|---|---|---|---|
| LOCUS | PRIMER SEQUENCE | SIZE | DINUCLEOTIDE | TAG |
| BAT 25 | Forward-TCG CCT CCA AGA ATG TAA GT | 110-125 | Mononucleotide | HEX |
| | Reverse-TCT GCA TTT TAA CTA TGG CTC | | | |
| BAT 26 | Forward-TGA CTA CTT TTG ACT TCA GCC | 107-125 | Mononucleotide | TET |
| | Reverse-AAC CAT TCA ACA TTT TAA ACC C | | | |
| D17S250 | Forward-GAA GTG ATG AAA AGT AAT TGA TC | 190-230 | Dinucleotide | FAM |
| | Reverse-GCT GGC CAT ATA TAT ATT TAA ACC | | | |
| D5S346 | Forward-ACT CAC TCT AGT GAT AAA TCG GG | 110-135 | Dinucleotide | FAM |
| | Reverse-AGC AGA TAA GAC AGT ATT ACT AGT T | | | |
| D2S123 | Forward-AAA CAG GAT GCC TGC CTT TA | 200-230 | Dinucleotide | TET |
| | Reverse-GGA CTT TCC ACC TAT GGG AC | | | |
| ACTC | Foward-CTT GAC CTG AAT GCA CTG TG | 70-98 | Dinucleotide | FAM |
| | Reverse-ATT CCA TAC CTG GGA ACG AG | | | |

TABLE 2
Second Panel of Markers for Evaluation of MSI in Colorectal Cancer

| | | PRODUCT | MONO OR | 5′ FLUORESCENT |
|---|---|---|---|---|
| LOCUS | PRIMER SEQUENCE | SIZE | DINUCLEOTIDE | TAG |
| BAT 40 | Forward-ATT AAC TTC CTA CAC CAC AAC | 110-140 | Mononucleotide | HEX |
| | Reverse-GTA GAG CAA GAG CAC CTT G | | | |
| MYG-L | Forward-TGG CGA GAG TCC ATC AAA G | 140-210 | Tetranucleotide | HEX |
| | Reverse-CCT TTT AAG CTG CAA CAA TTT G | | | |
| BAT 34G4 | Forward-ACC CTG GAG GAT TTC ATC TC | 120-145 | Mononucleotide | TET |
| | Reverse-AAC AAA GCG AGA CCC AGT CT | | | |
| D10S197 | Forward-ACC ACT GCA CTT CAG GTG ACA | 155-185 | Dinucleotide | TET |
| | Reverse-GTG ATA CTG TCC TCA GGT CTC C | | | |
| D18S55 | Forward-GGG AAG TCA AAT GCA AAA TG | 135-165 | Dinucleofide | FAM |
| | Reverse-AGC TTG TGA GTA ATC TTA TGC | | | |
| | TGT G | | | |

TABLE 3

| ATTEMPT # | BAT 25 | BAT 26 | D17S250 | D2S123 | D5S346 | ACTC |
|---|---|---|---|---|---|---|
| MULTIPLEX | 25ng and 50 ng DNA | | | | | Same PCR conditions as |
| FIRST ATTEMPT | 1.5 mM final MgCl | | | | | above-tried annealing at 55 |
| BAT 25, BAT 26, | 33 ng Primer | | | | | |
| D17S250, D2S123, | 0.4 mM final DNTP' | | | | | Tried multiplex conditions |
| D5S346 | 2 units Taq | | | | | 25 ng DNA |
| | | | | | | 1.5 mM final MgCl |
| ACTC Alone | 96 10 min | | | | | 33 ng Primer |
| | 96 10 sec - - - 45 cycles | | | | | 0.4 mM final DNTP's |
| | 55 30 sec - - - 45 cycles | | | | | 2 units Taq |
| | 70 30 sec - - - 45 cycles | | | | | Amplified very well |
| | 70 10 min | | | | | |
| | 4 forever | | | | | |
| | All amplified well except | Primers all amplified very well under these condition on their own. | | | | |
| | D17S250, D2S123 and | | | | | |
| | D5S346 were a little low | | | | | |
| MULTIPLEX | 25 ng DNA | 25 ng DNA | 25 ng DNA | 25 ng DNA | 25 ng DNA | 25 ng DNA |
| SECOND ATTEMPT | 1.5 mM final MgCl | 1.5 mM final MgCl | 1.5 mM final MgCl | 1.5 mM final MgCl | 1.5 mM final MgCl | 1.5 mM final MgCl |
| | 66 ng Primer F/840 ng R | 33 ng Primer | 45 ng Primer | 33 ng Primer | 33 ng Primer | 33 ng Primer |
| INCREASED F/R | 0.4 mM final DNTP's | 0.4 mM final DNTP's | 0.4 mM final DNTP's | 0.4 mM final DNTP's | 0.4 mM final DNTP's | 0.4 mM final DNTP's |
| PRIMERS FOR | 2 units Taq | 2 units Taq | 2 units Taq | 2 units Taq | 2 units Taq | 2 units Taq |
| BAT 25 | | | Tried extra forward and extra | | | |
| | Same PCR conditions as | Same PCR conditions as | forward and reverse | Same PCR conditions as | Same PCR conditions as | Same PCR conditions as |
| ADDED ACTC TO | above | above | | above | above | above |
| MULTIPLEX | | | Same PCR conditions as | | | |
| | | | above | | | |
| | Amplified very well | Amplified very well | Didn't amplify | Amplified weaker than the | Amplified weaker than the | Amplified very well |
| | | | | rest | rest | |
| MULTIPLEX | 25 ng DNA | 25 ng DNA | 25 ng DNA | 25 ng DNA | 25 ng DNA | 25 ng DNA |
| THIRD ATTEMPT | 1.5 mM final MgCl | 1.5 mM final MgCl | 1.5 mM final MgCl | 1.5 mM final MgCl | 1.5 mM final MgCl | 1.5 mM final MgCl |
| | 66 ng Primer F/840 ng R | 33 ng Primer | 33 ng Primer | 33ng Primer | 33 ng Primer | 33 ng Primer |
| BAT 25 WITH ALL | 0.4 mM final DNTP's | 0.4 mM final DNTP's | 0.4 mM final DNTP's | 0.4 mM final DNTP's | 0.4 mM final DNTP's | 0.4 mM final DNTP's |
| OTHER PRIMERS | 2 units Taq | 2 units Taq | 2 units Taq | 2 units Taq | 2 units Taq | 2 units Taq |
| EXCEPT D17S250 | | | | | | |
| | Same PCR conditions as | Same PCR conditions as | Same POR conditions as | Same PCR conditions as | Same PCR conditions as | Same PCR conditions as |
| | above | above | above | above | above | above |
| BAT 25 WITH | Amplified very well in | Amplified very well in | Amplified well in multiplex | Amplified well in multiple | Amplified well in multiplex | |
| | mult | multiplex | | | | |
| | | | Did not amplify with BAT 2 | Slightly weaker than rest | Slightly weaker than rest | |
| MULTIPLEX | 25 ng DNA | 25 ng DNA | | 25 ng DNA | 25 ng DNA | 25 ng DNA |
| FOURTH ATTEMPT | 1.5 mM final MgCl | 1.5 mM final MgCl | | 1.5 mM final MgCl | 1.5 mM final MgCl | 1.5 mM final MgCl |
| | 66 ng Primer F/840 ng R | 33 ng Primer | | 33 ng Primer F/420 ng R | 33 ng Primer F/420 ng R | 33 ng Primer |
| NO D17S250 | 0.4 mM final DNTP's | 0.4 mM final DNTP's | | 0.4 mM final DNTP's | 0.4 mM final DNTP's | 0.4 mM final DNTP's |
| | 2 units Taq | 2 units Taq | | 2 units Taq | 2 units Taq | 2 units Taq |
| INCREASED PRIMER | Same PCR conditions as | Same PCR conditions as | | Same PCR conditions as | Same PCR conditions as | Same PCR conditions as |
| RATIO TO 14:1 FOR | | | | | | |
| D2S123 AND D5S346 | above | above | | above | above | above |
| | D2S123 and D5S346 amplifed much better than previously seen. | | | | | |
| | All other loci amplified very well. | | | | | |
| FIFTH MULTIPLEX | 25 ng DNA | 25 ng DNA | | 25 ng DNA | 25 ng DNA | 25 ng DNA |
| ATTEMPT | 1.5 mM final MgCl | 1.5 mM final MgCl | | 1.5 mM final MgCl | 1.5 mM final MgCl | 1.5 mM final MgCl |
| | 66 ng Primer F/840 ng R | 33 ng Primer F/33 ng R | | 33 ng Primer F/420 ng R | 33 ng Primer F/ 420ng R | 33 ng Primer F/33 ng R |
| CHANGED TO | 0.4 mM final DNTP's | 0.4 mM final DNTP's | | 0.4 mM final DNTP's | 0.4 mM final DNTP's | 0.4 mM final DNTP's |
| PLATINUM TAQ | 3 units Taq | 3 units Taq | | 3 units Taq | 3 units Taq | 3 units Taq |
| USING MJ PCR | 94 5 min | 94 5 min | | 94 5 min | 94 5 min | 94 5 min |
| MACHINE | 94 30 sec - - - 40 cycles | 94 30 sec - - - 40 cycles | | 94 30 sec - - - 40 cycles | 94 30 sec - - - 40 cycles | 94 30 sec - - - 40 cycles |
| | 55 30 sec - - - 40 cycles | 55 30 sec - - - 40 cycles | | 55 30 sec - - - 40 cycles | 55 30 sec - - - 40 cycles | 55 30 sec - - - 40 cycles |
| | 72 45 sec - - - 40 cycles | 72 45 sec - - - 40 cycles | | 72 45 sec - - - 40 cycles | 72 45 sec - - - 40 cycles | 72 45 sec - - - 40 cycles |
| | 72 10 min | 72 10 min | | 72 10 min | 72 10 min | 72 10 min |
| | 4 forever | 4 forever | | 4 forever | 4 forever | 4 forever |
| | All loci are amplifiying very well | | | | | |
| FINAL MULTIPLEX | 25 ng DNA | 25 ng DNA | 25 ng DNA | 25 ng DNA | 25 ng DNA | 25 ng DNA |
| CONDITIONS | 1.5 mM final MgCl | 1.5 mM final MgCl | 1.5 mM final MgCl | 1.5 mM final MgCl | 1.5 mM final MgCl | 1.5 mM final MgCl |
| | 66 ng Primer F/840 ng R | 66 ng Primer F/66 ng R | 66 ng Primer F/66ng R | 66 ng Primer F/420 ng R | 33 ng Primer F/420 ng R | 33 ng Primer F/33 ng R |
| INCREASED BAT26 | 0.4 mM final DNTP's | 0.4 mM final DNTP's | 0.4 mM final DNTP's | 0.4 mM final DNTP's | 0.4 mM final DNTP's | 0.4 mM final DNTP's |
| F/R PRIMER CONC. | 3 units Taq | 3 units Taq | 3 units Taq | 3 units Taq | 3 units Taq | 3 units Taq |
| ADDED D175250 TO | 94 5 min | 94 5 min | 94 5 min | 94 5 min | 94 5 min | 94 5 min |
| MULTIPLEX | 94 30 sec - - - 35 cycles | 94 30 sec - - - 35 cycles | 94 30 sec - - - 40 cycles | 94 30 sec - - - 35 cycles | 94 30 sec - - - 35 cycles | 94 30 sec - - - 35 cycles |
| | 55 30 sec - - - 35 cycles | 55 30 sec - - - 35 cycles | 55 30 sec - - - 40 cycles | 55 30 sec - - - 35 cycles | 55 30 sec - - - 35 cycles | 55 30 sec - - - 35 cycles |
| INCREASED D2S123 | 72 30 sec - - - 35 cycles | 72 30 sec - - - 35 cycles | 72 30 sec - - - 40 cycles | 72 30 sec - - - 35 cycles | 72 30 sec - - - 35 cycles | 72 30 sec - - - 35 cycles |
| F PRIMER CONC. | 72 10 min | 72 10 min | 72 10 min | 72 10 min | 72 10 min | 72 10 min |
| | 4 forever | 4 forever | 4 forever | 4 forever | 4 forever | 4 forever |

| | |
|---|---|
| TWO STEP PCR | All loci are amplifying very well |

OVERALL RESULTS:
All Primers on their own will work with the MgCl at a final concentration of 1.5 mM to 4.0 mM.
All Primers on their own will work at an annealling temperature of 55 to 58 with BAT 25 and D17S250 working at 60 as well.
In a multiplex the loci will still amplify if the primers are at 33 ng or at 66 ng or higher (i.e. for reverse, 420 ng/840 ng)

TABLE 4

| ATTEMPT # | BAT 40 | BAT 34C4 | D10S197 | D18S55 | MYC-L |
|---|---|---|---|---|---|
| MULTIPLEX | 25 ng DNA | | | | |
| FIRST | 1.5 mM final MgCl | | | | |
| ATTEMPT | 33 ng F/R Primer | | | | |
| BAT 40, | 0.4 mM final DNTP's | | | | |
| BAT34C4 | | | | | |
| D10S197, | 2 units Taq | | | | |
| D18S55 | | | | | |
| MYC-L | | | | | |
| | 94 5 min | | | | |
| Increased | 94 30 sec - - - 40 cycles | | | | |
| elongationc | 55 30 sec - - - 40 cycles | | | | |
| to 45 sec | 72 45 sec - - - 40 cycles | | | | |
| | 72 10 min | | | | |
| | 4 forever | | | | |
| | | All primers amplified well together, slight background for BAT 40 | | | |
| MULTIPLEX | 25 ng DNA | | | | |
| SECOND | 1.5 mM final MgCl | | | | |
| ATTEMPT | 33 ng Primer | | | | |
| Increased | 0.4 mM F/R Primer | | | | |
| annealing | | | | | |
| temperature to 57 | 2 units Taq | | | | |
| | 94 5 min | | | | |
| | 94 30 sec - - - 40 cycles | | | | |
| | 57 30 sec - - - 40 cycles | | | | |
| | 72 45 sec - - - 40 cycles | | | | |
| | 72 10 min | | | | |
| | 4 forever | | | | |
| | | All primers amplified well, background for BAT 40 is greatly diminished | | | |
| FINAL | 25 ng DNA | 25 ng DNA | 25 ng DNA | 25 ng DNA | 25 ng DNA |
| MULTIPLEX | 1.5 mM final MgCl | 1.5 mM final MgCl | 1.5 mM final MgCl | 1.5 mM final MgCl | 1.5 mM final MgCl |
| CONDITIONS | 33 ng Primer | 33 ng Primer | 33 ng Primer | 33 ng Primer | 33 ng Primer |
| USING | 0.4 mM F/R Primer | 0.4 mM F/R Primer | 0.4 mM F/R Primer | 0.4 mM F/R Primer | 0.4 mM F/R Primer |
| PLATINUM | | | | | |
| TAQ | 2 units Taq | 2 units Taq | 2 units Taq | 2 units Taq | 2 units Taq |
| USING MJ PCR | 94 5 min | 94 5 min | 94 5 min | 94 5 min | 94 5 min |
| MACHINE | 94 30 sec - - - 40 cycles | 94 30 sec - - - 40 cycles | 94 30 sec - - - 40 cycles | 94 30 sec - - - 40 cycles | 94 30 sec - - - 40 cycles |
| | 57 30 sec - - - 40 cycles | 57 30 sec - - - 40 cycles | 57 30 sec - - - 40 cycles | 57 30 sec - - - 40 cycles | 57 30 sec - - - 40 cycles |
| | 72 45 sec - - - 40 cycles | 72 45 sec - - - 40 cycles | 72 45 sec - - - 40 cycles | 72 45 sec - - - 40 cycles | 72 45 sec - - - 40 cycles |
| | 72 10 min | 72 10 min | 72 10 min | 72 10 min | 72 10 min |
| | 4 forever | 4 forever | 4 forever | 4 forever | 4 forever |

All loci are amplifyIng very well

TABLE 5

| | | | | |
|---|---|---|---|---|
| LOCI | LOCATION | GENE NEAR MARKER | TYPE OF REPEAT | SIZE |

FIRST MULTIPLEX PANEL

| | | | | |
|---|---|---|---|---|
| BAT 25 | 4q12 | Intron of the C-kit | TTTT.T.TTTT.(T)₇.A(T)₂₅ | 110-125 |
| | | oncogene | | |
| BAT 26 | 2p | Intron 5 of MSH2 | (T)₅........(A)₂₆ | 105-125 |
| D5S346 | 5q21-22 | APC-a tumour | (CA)₂₈ | 110-140 |
| | | suppressor gene | | |
| D2S123 | 2p1 6 | MSH2 | (CA)₁₃TA(CA)₁₅(T/G A)₇ | 200-230 |
| ACTC | 15q11-qter | ACTC | (GT)₂₄ | 65-100 |
| D17S250 | 17q11.2-q12 | BRCA1 | (TA)₇...............(CA)₂₄ | 190-230 |

SECOND MULTIPLEX PANEL

| | | | | |
|---|---|---|---|---|
| BAT 40 | 1p13.1 | Intron of 3-B-hydroxysteroid | TTTT.TT.(T)₇..........TTTT.(T)₄₀ | 100-135 |
| | | dehydrogenase gene | | |
| BAT34C4 | | 11th exon of p53-repeat is | (T)₃C(T)₆C(T)₁₇C(T)₅C(T)₃ | 120-145 |
| | | in 3′ non-translated region | | |
| D10S197 | 10qter | | CACCAGA(CA)₇.A.A.(CA)₁₂(AGAAA)₂ | 155-185 |
| D18S55 | 18q22.1 | DCC-Deleted in Colorectal | (CA)₂₃ | 135-165 |
| | | Carcinomas | | |
| MYCL | 1p32 | MYCL | GAAAA(GAAA)₂TAAA(A/G)₁₀ | 140-210 |
| | | | GAAAGA(GAAA)₁₄GAAA(GAAAA)₈ | |
| | | | GAAAAA(GAAAA)₃ | |

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

We claim:

1. A method for evaluating microsatellite instability in a tumor sample by detecting microsatellite loci in the sample comprising:
(a) forming a polymerase chain reaction mixture comprising the tumor sample, a polymerase and primer sets for at least two selected microsatellite loci associated with cancer, each primer set characterized by (a) a forward primer containing a sequence complimentary to a 5′ upstream primer-specific portion of a selected microsatellite loci; and (b) a reverse primer complementary to a 3′ downstream primer-specific portion of the same microsatelite loci, wherein one of the primers has a detectable reporter label;
(b) subjecting the polymerase chain reaction mixture to polymerase chain reaction cycles to form amplified products complementary to microsatellite loci sequences in the tumor sample;
(c) detecting the reporter labels and distinguishing the amplified products to indicate the presence of one or more of the microsatellite loci in the sample; and
(d) repeating steps (a) to (c) with primer sets for at least two different selected microsatellite loci.

2. A method as claimed in claim 1 wherein in step (b) the polymerase chain reaction cycles comprise a denaturation treatment, wherein hybridized nucleic acid sequences are separated, a hybridization treatment, wherein the primers hybridize to their complementary primer-specific portions of a microsatellite loci sequence, and an extension treatment, wherein the hybridized primers are extended.

3. A method as claimed in claim 1 wherein in step (a) the primers are for the BAT26 and D17S250 loci, and optionally one or more of BAT25, D5S346, D2S123, and ACTC loci.

4. A method as claimed in claim 1 wherein in step (a) the primers are the primers in Table 1.

5. A method as claimed in claim 1 wherein in step (b) the primers are for MYC-L, one or both of BAT40 and BAT34C4, and optionally one or both of D10S197 and D18S55.

6. A method as claimed in claim 1 wherein in step (b) wherein the primers are the primers in Table 2.

7. A method as claimed in claim 1 wherein the detectable reporter label is a chromophore, fluorescent molecule, enzyme, antigen, heavy metal, magnetic probe, dye, radioactive material, phosphorescent group, chemiluminescent moiety, or electrochemical detecting moiety.

8. A method as claimed in claim 1 wherein the tumor sample is a body tissue or fluid suitable for detecting tumor cells.

9. A method as claimed in claim 1 wherein the tumor sample comprises nucleic acids.

10. A method as claimed in claim 9 wherein the nucleic acids are present in the tumor sample at a concentration of 20-75 ng.

11. A method as claimed in claim 1 wherein the concentration of the forward primer is about 25-65 ng and the concentration of the reverse primer is about 30-840 ng.

12. A method as claimed in claim 1 wherein the cancer involves defects in mismatch repair of genes.

13. A method as claimed in claim 1 wherein the cancer is leukemia, colorectal cancer, breast cancer, lung cancer, prostate cancer, brain tumors, central nervous system tumors, bladder tumors, melanomas, liver cancer, bone cancer, testicular carcinoma, ovarian carcinoma, head and neck tumors, or cervical cancer.

14. A method as claimed in claim 1 wherein the cancer is colorectal cancer or hereditary non-polyposis colorectal cancer syndrome.

15. A method for diagnosing colorectal cancer or hereditary non-polyposis colorectal cancer syndrome in a human individual comprising the steps of (a) isolating DNA from the human individual; (b) assaying the DNA using multiplex polymerase chain reaction for microsatellite loci associated with colorectal cancer or hereditary non-polyposis colorectal cancer syndrome relative to a normal human individual (c) diagnosing colorectal cancer or hereditary non-polyposis colorectal cancer syndrome in the human individual based on the frequency of microsatellite loci.

16. A method as claimed in claim 15 wherein at least 6, 8, or 10 microsatellite loci are assayed.

17. A method as claimed in claim 15 wherein the microsatellite loci assayed are BAT26, D17S250, MYC-L, one or both of BAT40, and optionally one or more of BAT25, D5S346, D2S123, ACTC, D10S197 and D18S55.

18. A method as claimed in claim 15 wherein the microsatellite loci assayed are the loci identified in Table 1 and Table 2.

19. A kit comprising compositions selected from the group consisting of primers and ancillary reagents used in a polymerase chain reaction in a method as claimed in claim 1.

20. A kit comprising compositions selected from the group consisting of primers and ancillary reagents used in a mulitplex olymerase chain reaction in a method as claimed in claim 15.
